(19) United States
(12) Patent Application Publication
(10) Pub. No.: US 2020/0181681 A1
(43) Pub. Date: Jun. 11, 2020

(54) MULTIPLEXED OPTIMIZED MISMATCH AMPLIFICATION (MOMA)-REAL TIME PCR FOR ASSESSING CANCER

(71) Applicant: The Medical College of Wisconsin, Inc., Milwaukee, WI (US)

(72) Inventors: Aoy Tomita Mitchell, Elm Grove, WI (US); Karl Stamm, Wauwatosa, WI (US)

(73) Assignee: The Medical College of Wisconsin, Inc., Milvaukee, WI (US)

(21) Appl. No.: 16/097,404

(22) PCT Filed: Apr. 29, 2017
(86) PCT No.: PCT/US2017/030291

§ 371 Date: Oct. 29, 2018

Related U.S. Application Data
(60) Provisional application No. 62/330,043, filed on Apr. 29, 2016.

(51) Int. Cl. C12Q 1/686; C12Q 1/6886; C12Q 1/6858; C12Q 1/6806

(52) U.S. Cl. CPC C12Q 1/686; C12Q 1/6886; C12Q 2600/156; C12Q 1/6806; C12Q 1/6858

(57) ABSTRACT

This invention relates to methods and compositions for assessing an amount of cancer-specific nucleic acids in a sample, such as from a subject. The methods and compositions provided herein can be used to determine risk of a condition, such as cancer, in a subject.

### RELATED APPLICATIONS

This application claims the benefit under 35 U.S.C. § 119(e) of the filing date of U.S. Provisional Application 62/330,043, filed Apr. 29, 2016, the contents of which are incorporated herein by reference in their entirety.

### FIELD OF THE INVENTION

This invention relates to methods and compositions for assessing an amount of non-native nucleic acids in a sample from a subject. The methods and compositions provided herein can be used to determine risk of a condition, such as cancer. This invention further relates to methods and compositions for assessing the amount of non-native cell-free deoxyribonucleic acid (non-native cell-free DNA, such as cancer-specific cell-free DNA) using multiplexed optimized mismatch amplification (MOMA).

### SUMMARY OF THE INVENTION

The present disclosure is based, at least in part on the surprising discovery that multiplexed optimized mismatch amplification can be used to quantify low frequency non-native nucleic acids in samples from a subject. Multiplexed optimized mismatch amplification embraces the design of primers that can include a 3′ penultimate mismatch for the amplification of a specific sequence but a double mismatch relative to an alternate sequence. Amplification with such primers can permit the quantitative determination of amounts of non-native nucleic acids in a sample, even where the amount of non-native nucleic acids are, for example, below 1%, or even 0.5%, in a heterogeneous population of nucleic acids.

Provided herein are methods, compositions and kits related to such amplification assays. The methods, compositions or kits can be any one of the methods, compositions or kits, respectively, provided herein, including any one of those of the examples and drawings.

In one aspect, a method of assessing an amount of cancer-specific nucleic acids in a sample from a subject is provided. In one embodiment the method comprises, for each of one or more single nucleotide variant (SNV) targets, performing an amplification-based quantification assay, such as a polymerase chain reaction (PCR) quantification assay, on the sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target, and obtaining or providing results from the amplification-based quantification assays, such as PCR quantification assays, to determine the amount of cancer-specific nucleic acids in the sample.

In one embodiment of any one of the methods, compositions or kits provided herein, the results are provided in a report.

In one embodiment of any one of the methods provided herein, the method further comprises determining the amount of the cancer-specific nucleic acids in the sample based on the results. In one embodiment of any one of the methods, compositions or kits provided herein, the results comprise the amount of the cancer-specific nucleic acids in the sample.

In one aspect, a method of assessing an amount of cancer-specific nucleic acids in a sample from a subject, the method comprising obtaining results from an amplification-based quantification assay, such as a polymerase chain reaction (PCR) quantification assay, for each of one or more single nucleotide variant (SNV) targets, performed on the sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target and specifically amplifies the one allele of the SNV target in a primer, and another of the at least two primer pairs specifically amplifies the another allele of the SNV target, and assessing the amount of cancer-specific nucleic acids based on the results is provided.

In one embodiment of any one of the methods, compositions or kits provided herein, the amount of the cancer-specific nucleic acids in the sample is based on the results of the amplification-based quantification assays, such as PCR quantification assays. In one embodiment of any one of the methods, compositions or kits provided herein, the results are obtained from a report.

In one embodiment of any one of the methods, compositions or kits provided herein, the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

In one embodiment of any one of the methods, compositions or kits provided herein, the amount is the ratio or percentage of cancer-specific nucleic acids to wild-type or total nucleic acids as measured in the assay.

In one embodiment of any one of the methods, compositions or kits provided herein, the results are informative results of the amplification-based quantification assays, such as PCR quantification assays. In one embodiment of any one of the methods, compositions or kits provided herein, the amount is based on informative results of the amplification-based quantification assays, such as PCR quantification assays.

In one embodiment of any one of the methods provided herein, the method further comprises selecting informative results of the amplification-based quantification assays, such as PCR quantification assays. In one embodiment of any one of the methods, compositions or kits provided herein, the selected informative results are averaged.

In one embodiment of any one of the methods provided herein, the informative results of the amplification-based quantification assays, such as PCR quantification assays, are selected based on the genotype of the subject. In one embodiment of any one of the methods provided herein, the method further comprises obtaining the genotype of the subject.

In one embodiment of any one of the methods provided herein, the method further comprises obtaining the plurality of SNV targets. In one embodiment of any one of the methods provided herein, the method further comprises obtaining the at least two primer pairs for each of the one or more SNV targets.

In one embodiment of any one of the methods, compositions or kits provided herein, the one or more SNV targets is at least 1 SNV target. In one embodiment of any one of the methods, compositions or kits provided herein, the one or more SNV targets is at least 2 SNV targets. In one embodiment of any one of the methods, compositions or kits provided herein, the one or more SNV targets is at least 3 SNV targets. In one embodiment of any one of the methods, compositions or kits provided herein, the one or more SNV targets is at least 4 SNV targets. In one embodiment of any one of the methods, compositions or kits provided herein, the one or more SNV targets is at least 5 SNV targets. In one embodiment of any one of the methods, compositions or kits provided herein, the one or more SNV targets is at least 6 SNV targets. In one embodiment of any one of the methods, compositions or kits provided herein, the one or more SNV targets is at least 7 SNV targets. In one embodiment of any one of the methods, compositions or kits provided herein, the one or more SNV targets is at least 8 SNV targets. In one embodiment of any one of the methods, compositions or kits provided herein, the one or more SNV targets is at least 9 SNV targets. In one embodiment of any one of the methods, compositions or kits provided herein, the one or more SNV targets is at least 10 SNV targets. In one embodiment of any one of the methods, compositions or kits provided herein, the one or more SNV targets is at least 11 SNV targets. In one embodiment of any one of the methods, compositions or kits provided herein, the one or more SNV targets is at least 12 SNV targets. In one embodiment of any one of the methods, compositions or kits provided herein, the one or more SNV targets is at least 13 SNV targets. In one embodiment of any one of the methods, compositions or kits provided herein, the one or more SNV targets is at least 14 SNV targets. In one embodiment of any one of the methods, compositions or kits provided herein, the one or more SNV targets is at least 15 SNV targets.

In one embodiment of any one of the methods, compositions or kits provided herein, the one or more SNV targets are each specific to the same kind of cancer. In one embodiment of any one of the methods, compositions or kits, the kind of cancer is pancreatic cancer. In one embodiment of any one of the methods, compositions or kits, the one or more SNV targets comprise a SNV target in the KRAS gene and/or p53 gene. In one embodiment of any one of the methods, compositions or kits, the one or more SNV targets are each specific to a cancer in the subject. In one embodiment of any one of the methods, compositions or kits, at least one SNV target is specific to one kind of cancer and at least one other SNV target is specific to another kind of cancer. In one embodiment of any one of the methods provided herein, the SNV targets are sequences mutated in the subject's prior cancer.

In one embodiment of any one of the methods provided herein, the method further comprises obtaining the genotype of the cancer in the subject.

In one embodiment of any one of the methods provided herein, the amount of cancer-specific nucleic acids in the sample is at least 0.25%. In one embodiment of any one of the methods provided herein, the amount of cancer-specific nucleic acids in the sample is at least 0.5%. In one embodiment of any one of the methods provided herein, the amount of cancer-specific nucleic acids in the sample is at least 1%. In one embodiment of any one of the methods provided herein, the amount of cancer-specific nucleic acids in the sample is at least 2%. In one embodiment of any one of the methods provided herein, the amount of cancer-specific nucleic acids in the sample is at least 5%.

In one embodiment of any one of the methods provided herein, the cancer-specific nucleic acids are cancer-specific cell-free DNA.

In one embodiment of any one of the methods provided herein, the PCR quantification assays are real time PCR assays or digital PCR assays.

In one embodiment of any one of the methods provided herein, the method further comprises determining a risk in the subject based on the amount of cancer-specific nucleic acids in the sample. In one embodiment of any one of the methods provided herein, the risk is a risk associated with cancer. In one embodiment of any one of the methods provided herein, the risk is increased if the amount of cancer-specific nucleic acids is greater than a threshold value. In one embodiment of any one of the methods provided herein, the risk is decreased if the amount of cancer-specific nucleic acids is less than a threshold value.

In one embodiment of any one of the methods provided herein, the method further comprises selecting a treatment for the subject based on the amount of cancer-specific nucleic acids. In one embodiment of any one of the methods provided herein, the method further comprises treating the subject based on the amount of cancer-specific nucleic acids.

In one embodiment of any one of the methods provided herein, the method further comprises providing information about a treatment to the subject based on the amount of cancer-specific nucleic acids.

In one embodiment of any one of the methods provided herein, the method further comprises monitoring or suggesting the monitoring of the amount of cancer-specific nucleic acids in the subject over time or at a subsequent point in time. In one embodiment of any one of the methods provided herein, the method further comprises evaluating an effect of a treatment administered to the subject based on the amount of cancer-specific nucleic acids. In one embodiment of any one of the methods provided herein, the treatment is a cancer treatment.

In one embodiment of any one of the methods provided herein, the method further comprises providing or obtaining the sample or a portion thereof. In one embodiment of any one of the methods provided herein, the method further comprises extracting nucleic acids from the sample. In one embodiment of any one of the methods provided herein, the method further comprising performing a pre-amplification step using primers for the SNV targets. The primers may be the same or different as those for determining the amount of non-native nucleic acids.

In one embodiment of any one of the methods provided herein, the probe in one or more or all of the PCR quantification assays is on the same strand as the mismatch primer and not on the opposite strand.

In one embodiment of any one of the methods provided herein, the sample comprises blood, plasma or serum.

In one aspect, a composition or kit comprising, a primer pair, for each of one or more cancer-specific SNV targets, wherein each primer pair comprises a 3′ penultimate mismatch relative to one allele of a SNV target but a 3′ double mismatch relative to another allele of the SNV target in a primer and specifically amplifies the one allele of the SNV target, wherein the one or more SNV targets is provided.

In one embodiment of any one of the compositions or kits provided herein, the composition or kit further comprises another primer pair for each of the one or more cancer-specific SNV targets wherein the another primer pair specifically amplifies the another allele of the SNV target.

In one embodiment of any one of the methods, compositions or kits provided herein, the one or more cancer-specific SNV targets is at least 2, 3, 4, 5, 6, 7, 8, 9, 10, 11, 12, 13, 14 or 15 SNV targets. In one embodiment of any one of the methods, compositions or kits provided herein, the cancer-specific SNV targets are each specific to the same kind of cancer. In one embodiment of any one of the methods, compositions or kits provided herein, the kind of cancer is pancreatic cancer. In one embodiment of any one of the methods, compositions or kits provided herein, the cancer-specific SNV targets comprise a SNV target in the KRAS gene and/or p53 gene.

In one embodiment of any one of the methods, compositions or kits provided herein, the cancer-specific SNV targets are each specific to a cancer in the subject. In one embodiment of any one of the methods, compositions or kits provided herein, at least one SNV target is specific to one kind of cancer and at least one other SNV target is specific to another kind of cancer.

In one embodiment of any one of any one of the methods, compositions or kits provided herein, the another primer pair for each of the SNV targets also comprises a 3′ penultimate mismatch relative to the another allele of the SNV target but a 3′ double mismatch relative to the one allele of the SNV target in a primer and specifically amplifies the another allele of the SNV target.

In one embodiment of any one of the compositions or kits provided herein, the composition or kit further comprises a buffer. In one embodiment of any one of the compositions or kits provided herein, the composition or kit further comprises a polymerase. In one embodiment of any one of the compositions or kits provided herein, the composition or kit further comprises a probe. In one embodiment of any one of the compositions or kits provided herein, the probe is a fluorescent probe.

In one embodiment of any one of the compositions or kits provided herein, the composition or kit further comprises instructions for use. In one embodiment of any one of the compositions or kits provided herein, the instructions for use are instructions for determining or assessing the amount of cancer-specific nucleic acids in a sample from a subject at risk of cancer, with cancer or suspected of having cancer.

In one aspect, any one of the compositions or kits provided herein can be for use any one of the methods provided herein.

In one aspect, a method comprising obtaining the amount of cancer-specific nucleic acids based on any one of the methods provided herein, and assessing a risk in a subject that is at risk of cancer, has cancer, is suspected of having cancer or previously had cancer based on the levels or amount is provided.

In one embodiment of any one of the methods provided herein, a treatment or information about a treatment or non-treatment is selected for or provided to the subject based on the assessed risk. In one embodiment of any one of the methods provided herein, the method further comprises monitoring or suggesting the monitoring of the amount of cancer-specific nucleic acids in the subject over time. In one embodiment of any one of the methods provided, the method further comprises obtaining another sample from the subject, such as at a subsequent point in time, and performing a test on the sample, such as any one of the methods provided herein.

In one aspect, a report containing one or more of the results as provided herein is provided. In one embodiment of any one of the reports provided, the report is in electronic form. In one embodiment of any one of the reports provided, the report is a hard copy. In one embodiment of any one of the reports provided, the report is given orally.

In one embodiment of any one of the methods, compositions or kits provided, the mismatched primer(s) is/are the forward primer(s). In one embodiment of any one of the methods, compositions or kits provided, the reverse primers for the primer pairs for each SNV target is the same.

In one embodiment, any one of the embodiments for the methods provided herein can be an embodiment for any one of the compositions, kits or reports provided. In one embodiment, any one of the embodiments for the compositions, kits or reports provided herein can be an embodiment for any one of the methods provided herein.

### BRIEF DESCRIPTION OF DRAWINGS

The accompanying drawings are not intended to be drawn to scale. The figures are illustrative only and are not required for enablement of the disclosure.

FIG. 1 provides an exemplary, non-limiting diagram of MOMA primers. In a polymerase chain reaction (PCR) assay, extension of the sequence containing SNV A is expected to occur, resulting in the detection of SNV A, which may be subsequently quantified. Extension of the SNV B, however, is not expected to occur due to the double mismatch.

FIG. 2 provides exemplary amplification traces.

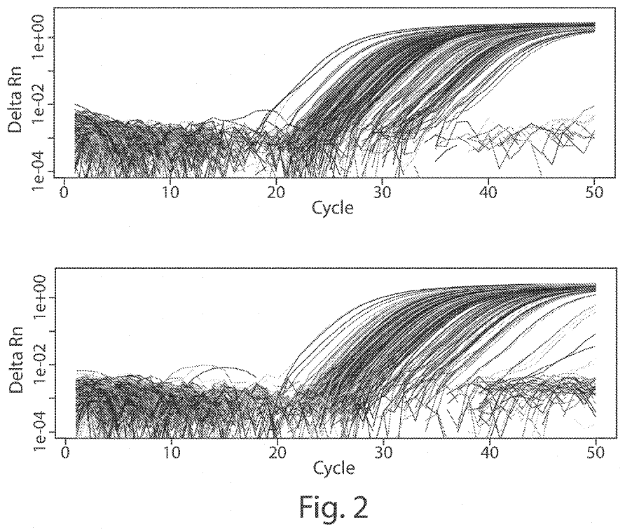

FIG. 3 provides the average background noise for 104 MOMA targets.

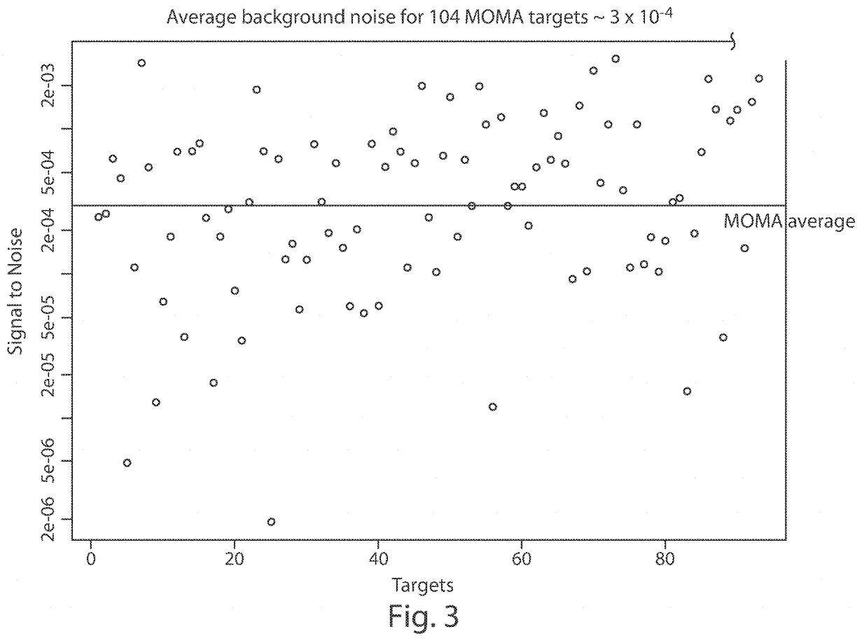

FIG. 4 provides further examples of the background noise for methods using MOMA.

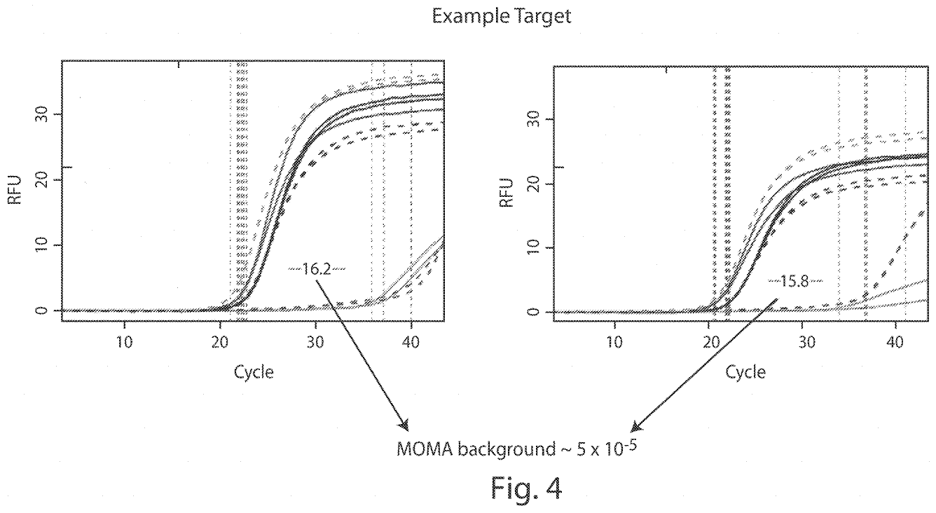

FIG. 5 provides amplification curves and the standard curve as described in the Examples.

FIG. 6 illustrates an example of a computer system with which some embodiments may operate.

### DETAILED DESCRIPTION OF THE INVENTION

Aspects of the disclosure relate to methods for the sensitive detection and/or quantification of non-native nucleic acids in a sample. Non-native nucleic acids, such as non-native DNA, may be present in individuals in a variety of situations including cancer. The disclosure provides techniques to detect, analyze and/or quantify non-native nucleic acids, such as non-native cell-free DNA concentrations, in samples obtained from a subject, such as those at risk for, or those with, cancer.

As used herein, “non-native nucleic acids” refers to nucleic acids that are from another source or are mutated versions of a nucleic acid found in a subject (with respect to a specific sequence, such as a wild-type (WT) sequence). “Native nucleic acids”, therefore, are nucleic acids that are not from another source and are not mutated versions of a nucleic acid found in a subject (with respect to a specific sequence). In some embodiments, the non-native nucleic acid is non-native cell-free DNA. “Cell-free DNA” (or cf-DNA) is DNA that is present outside of a cell, e.g., in the blood, plasma, serum, urine, etc. of a subject. Without wishing to be bound by any particular theory or mechanism, it is believed that cf-DNA is released from cells, e.g., via apoptosis of the cells. An example of non-native nucleic acids are nucleic acids that are from a cancer in a subject. As used herein, the compositions and methods provided herein can be used to determine an amount of cell-free DNA from a non-native source, such as DNA specific to a cancer or cancer-specific cell-free DNA (e.g., cancer-specific cfDNA, CS cfDNA).

Provided herein are methods and compositions that can be used to measure nucleic acids with differences in sequence identity. In some embodiments, the difference in sequence identity is a single nucleotide variant (SNV); however, wherever a SNV is referred to herein any difference in sequence identity between native and non-native nucleic acids is intended to also be applicable. Thus, any one of the methods or compositions provided herein may be applied to native versus non-native nucleic acids where there is a difference in sequence identity. As used herein, “single nucleotide variant” refers to a nucleic acid sequence within which there is sequence variability at a single nucleotide. These SNVs can be known cancer mutations or any mutations specific to or that can identify a cancer. In some embodiments of any one of the methods provided herein, such mutations are mutations associated with any one of the cancers provided herein. In some embodiments of any one of the methods provided herein, the mutations are mutations from a cancer the subject had at one time, and the methods are methods for monitoring the subject for recurrence of the cancer. Primers can be prepared as provided herein for any one or more of the mutations provided.

Examples of genes in which cancer associated mutations can occur include tumor suppressor genes, such as, but not limited to, ARHGEF12, ATM, BCL11B, BLM, BMPR1A, BRCA1, BRCA2, CARS, CBFA2T3, CDH1, CDK6, CDKN2C, CEBPA, CHEK2, CREB1, CREBBP, CYLD, DDX5, EXT1, EXT2, FBXW7, FH, FLT3, FOXP1, GPC3, IDH1, IL2, JAK2, MAP2K4, MDM4, MEN1, MLH1, MSH2, NF1, NF2, NOTCH1, NPM1, NR4A3, NUP98, PALB2, PML, PTEN, RB1, RUNX1, SDHB, SDHD, SMARCA4, SCARCB1, SOCS1, STK11, SUFU, SUZ12, SYK, TCF3, TNFAIP3, TP53, TSC1, TSC2, WRN, WT1, pVHL, APC, CD95, ST5, YPEL3, ST7, and ST14. Other examples are oncogenes and include, but are not limited to, ABL1, ABL2, AKT1, AKT2, ATF1, BCL11A, BCL2, BCL3, BCL6, BCR, BRAF, CARD11, CBLB, CBLC, CCND1, CCND2, CCND3, CDX2, CTNNB1, DDB2, DDIT3, DDX6, DEK, EGFR, ELK4, ERBB2, ETV4, ETV6, EVI1, EWSR1, FEV, FGFR1, FGFR1OP, FGFR2, FUS, GOLFA5, GOPC, HMGA1, HMGA2, HRAS, IRF4, JUN, KIT, KRAS, LCK, LMO2, MAF, MAFB, MAML2, MDM2, MET, MITF, MLL, MPL, MYB, MYCL1, MYCN, NCOA4, NFKB2, NRAS, NTRK1, NUP214, PAX8, PDGFB, PIK3CA, PIM1, PLAG1, PPARG, PTPN11, RAF1, REL, RET, ROS1, SMO, SS18, TCL1A, TET2, TFG, TLX1, PR, USP6, RAS, WNT, MYC, ERK, and TRK. The SNV targets as provided herein may be mutant sequences of any one or more of these genes in some embodiments.

The nucleic acid sequence within which there is sequence identity variability, such as a SNV, is generally referred to as a “target”. As used herein, a “SNV target” refers to a nucleic acid sequence within which there is sequence variability, such as at a single nucleotide. The SNV target has more than one allele, and in preferred embodiments, the SNV target is biallelic. It has been discovered that non-native nucleic acids can be quantified even at extremely low levels by performing amplification-based quantification assays, such as quantitative PCR assays, with primers specific for SNV targets. In some embodiments, the amount of non-native nucleic acids is determined by attempting an amplification-based quantification assay, such as quantitative PCR, with primers for a plurality of SNV targets. A “plurality of SNV targets” refers to more than one SNV target where for each target there are at least two alleles. Preferably, in some embodiments, each SNV target is expected to be biallelic and a primer pair specific to each allele of the SNV target is used to specifically amplify nucleic acids of each allele, where amplification occurs if the nucleic acid of the specific allele is present in the sample. In some embodiments of any one of the methods provided herein, an amplification-based quantification assay, such as quantitative PCR, is performed with primer pairs for at least 1, 2, 3, 4, 5, 6, 7, 8, 9, 10, 11, 12, 13, 14 or 15 targets. In some embodiments of any one of the methods provided herein, an amplification-based quantification assay, such as quantitative PCR, is performed with primer pairs for at least 1, 2, 3, 4, 5, 6, 7, 8, 9, or 10 but less than 15 targets. In some embodiments of any one of the methods provided herein, an amplification-based quantification assay, such as quantitative PCR, is performed with primer pairs for at least 2, 3, 4, 5, 6, 7, 8, 9, 10, 11, 12, 13, 14 or 15 targets. In some embodiments of any one of the methods provided herein, an amplification-based quantification assay, such as quantitative PCR, is performed with primer pairs for at least 2, 3, 4, 5, 6, 7, 8, 9 or 10 but less than 15 targets. As used herein, one allele may be the mutated version of a target sequence and another allele is the non-mutated version of the sequence.

In an embodiment of any one of the methods or compositions provided herein, one or more primer pairs for SNV target(s) can be pre-selected based on knowledge that the SNV targets will be informative, such as with knowledge of genotype, such as of the cancer. In such embodiments, the subject may have previously had cancer and the method is for assessing the recurrence of the cancer. In such embodiments, the subject may have been previously been diagnosed with the cancer, and the method is for monitoring the cancer over time. In such embodiments, the genotype of the cancer is determined. Thus, any one of the methods provided herein, can include a step of genotyping the cancer in the subject or obtaining the genotype.

In another embodiment of any one of the methods or compositions provided herein, primer pairs for a plurality of SNV targets are selected for the likelihood at least one may be informative. In such embodiments, primer pairs for a panel of cancer-specific SNV targets is used in any one of the methods provided herein. In some embodiments, each of the panel of cancer-specific SNV targets are specific to the same kind of cancer. The cancer can be any one of the cancers as provided herein or otherwise known in the art. The SNV target may be one in any one of the cancer-associated genes as provided herein or otherwise known in the art. In other embodiments, the panel is directed to a number of SNV targets specific to a number of different kinds of cancer, one or more specific to one kind of cancer and one or more specific to another kind of cancer, etc. In some embodiments, such a panel can be directed to a number of the more common SNV targets associated with a number of the more common cancers.

For any one of the methods or compositions provided, the method or composition can be directed to any one of the foregoing numbers of targets.

As used herein, “an informative SNV target” is one in which amplification with primers as provided herein occurs, and the results of which are informative. “Informative results” as provided herein are the results that can be used to quantify the level of non-native and/or native nucleic acids in a sample. In some embodiments, informative results exclude results that are considered “no call” or erroneous call results. From the informative results, allele percentages can be calculated using standard curves, in some embodiments of any one of the methods provided. In some embodiments of any one of the methods provided, the amount of non-native and/or native nucleic acids represents an average across informative results for the non-native and/or native nucleic acids, respectively.

The amount or level, such as ratio or percentage, of non-native nucleic acids may be determined with the quantities of the major and minor alleles as well as the genotype of the native nucleic acids in some embodiments. In some embodiments of any one of the methods provided herein, the alleles can be determined based on prior genotyping of the native nucleic acids of the subject. Methods for genotyping are well known in the art. Such methods include sequencing, such as next generation, hybridization, microarray, other separation technologies or PCR assays. Any one of the methods provided herein can include steps of obtaining such genotypes.

“Obtaining” as used herein refers to any method by which the respective information or materials can be acquired. Thus, the respective information can be acquired by experimental methods, such as to determine the native genotype, in some embodiments. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

Reports may be in oral, written (or hard copy) or electronic form, such as in a form that can be visualized or displayed. In some embodiments, the “raw” results for each assay as provided herein are provided in a report, and from this report, further steps can be taken to determine the amount of non-native nucleic acids in the sample. These further steps may include any one or more of the following, selecting informative results, obtaining the native genotype, calculating allele percentages for informative results for the native and non-native nucleic acids, averaging the allele percentages, etc. In other embodiments, the report provides the amount of non-native nucleic acids in the sample. From the amount, in some embodiments, a clinician may assess the need for a treatment for the subject or the need to monitor the amount of the non-native nucleic acids over time. Accordingly, in any one of the methods provided herein, the method can include assessing the amount of non-nucleic acids in the subject at more than one point in time. Such assessing can be performed with any one of the methods or compositions provided herein.

In some embodiments, any one of the methods provided herein may include a step of determining or obtaining the total amount of nucleic acids, such as total cell-free DNA, in one or more samples from the subject. Accordingly, any one or more of the reports provided herein may also include one or more amounts of the total nucleic acids, such as total cell-free DNA, and it is the combination of the amount of non-native nucleic acids and total nucleic acids that is in a report and from which a clinician may assess the need for a treatment for the subject or the need to monitor the subject.

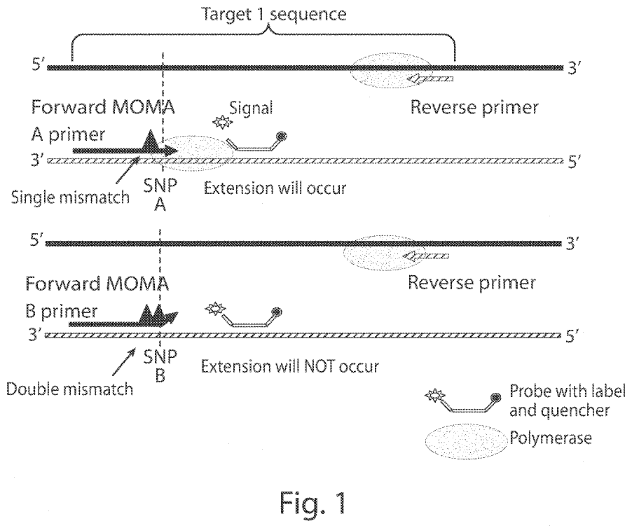

The amplification-based quantification assays, such as PCR assays, as provided herein make use of multiplexed optimized mismatch amplification (MOMA). Primers for use in such assays may be obtained, and any one of the methods provided herein can include a step of obtaining one or more primer pairs for performing the amplification-based quantification assays, such as PCR assays. Generally, the primers possess unique properties that facilitate their use in quantifying amounts of nucleic acids. For example, a forward primer of a primer pair can be mismatched at a 3′ nucleotide (e.g., penultimate 3′ nucleotide). In some embodiments of any one of the methods or compositions provided, this mismatch is at a 3′ nucleotide but adjacent to the SNV position. In some embodiments of any one of the methods or composition provided, the mismatch positioning of the primer relative to a SNV position is as shown in FIG. 1. Generally, such a forward primer even with the 3′ mismatch to produce an amplification product (in conjunction with a suitable reverse primer) in an amplification reaction, such as a PCR reaction, thus allowing for the amplification and resulting detection of a nucleic acid with the respective SNV. If the particular SNV is not present, and there is a double mismatch with respect to the other allele of the SNV target, an amplification product will generally not be produced. Preferably, in some embodiments of any one of the methods or compositions provided herein, for each SNV target a primer pair is obtained whereby specific amplification of each allele can occur without amplification of the other allele(s). “Specific amplification” refers to the amplification of a specific allele of a target without substantial amplification of another nucleic acid or without amplification of another nucleic acid sequence above background or noise. In some embodiments, specific amplification results only in the amplification of the specific allele.

In some embodiments of any one of the methods or compositions provided herein, for each SNV target that is biallelic, there are two primer pairs, each specific to one of the two alleles and thus have a single mismatch with respect to the allele it is to amplify and a double mismatch with respect to the allele it is not to amplify (again if nucleic acids of these alleles are present). In some embodiments of any one of the methods or compositions provided herein, the mismatch primer is the forward primer. In some embodiments of any one of the methods or compositions provided herein, the reverse primer of the two primer pairs for each SNV target is the same.

These concepts can be used in the design of primer pairs for any one of the compositions and methods provided herein. It should be appreciated that the forward and reverse primers are designed to bind opposite strands (e.g., a sense strand and an antisense strand) in order to amplify a fragment of a specific locus of the template. The forward and reverse primers of a primer pair may be designed to amplify a nucleic acid fragment of any suitable size to detect the presence of, for example, an allele of a SNV target according to the disclosure. Any one of the methods provided herein can include one or more steps for obtaining one or more primer pairs as described herein.

It should be appreciated that the primer pairs described herein may be used in a multiplex amplification-based quantification assay, such as a PCR assay. Accordingly, in some embodiments of any one of the methods or compositions provided herein, the primer pairs are designed to be compatible with other primer pairs in a PCR reaction. For example, the primer pairs may be designed to be compatible with at least 1, at least 2, at least 3, at least 4, at least 5, etc. other primer pairs in a PCR reaction. As used herein, primer pairs in a PCR reaction are “compatible” if they are capable of amplifying their target in the same PCR reaction. In some embodiments, primer pairs are compatible if the primer pairs are inhibited from amplifying their target DNA by no more than 1%, no more than 2%, no more than 3%, no more than 4%, no more than 5%, no more than 10%, no more than 15%, no more than 20%, no more than 25%, no more than 30%, no more than 35%, no more than 40%, no more than 45%, no more than 50%, or no more than 60% when multiplexed in the same PCR reaction. Primer pairs may not be compatible for a number of reasons including, but not limited to, the formation of primer dimers and binding to off-target sites on a template that may interfere with another primer pair. Accordingly, the primer pairs of the disclosure may be designed to prevent the formation of dimers with other primer pairs or limit the number of off-target binding sites. Exemplary methods for designing primers for use in a multiplex PCR assay are known in the art or otherwise described herein.

In some embodiments, the primer pairs described herein are used in a multiplex amplification-based quantification assay, such as a PCR assay, to quantify an amount of non-native nucleic acids. Accordingly, in some embodiments of any one of the methods or compositions provided herein, the primer pairs are designed to detect genomic regions that are diploid, excluding primer pairs that are designed to detect genomic regions that are potentially non-diploid. In some embodiments of any one of the methods or compositions provided herein, the primer pairs used in accordance with the disclosure do not detect repeat-masked regions, known copy-number variable regions, or other genomic regions that may be non-diploid.

In some embodiments of any one of the methods provided herein, the amplification-based quantitative assay is any quantitative assay, such as whereby nucleic acids are amplified and the amounts of the nucleic acids can be determined. Such assays include those whereby nucleic acids are amplified with the MOMA primers as described herein and quantified. Such assays include simple amplification and detection, hybridization techniques, separation technologies, such as electrophoresis, next generation sequencing and the like.

In some embodiments of any one of the methods provided herein the PCR is quantitative PCR meaning that amounts of nucleic acids can be determined. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

A probe may be useful for methods of the present disclosure, particularly for those methods that include a quantification step. Any one of the methods provided herein can include the use of a probe in the performance of the PCR assay(s), while any one of the compositions or kits provided herein can include one or more probes. Importantly, in some embodiments of any one or more of the methods provided herein, the probe in one or more or all of the PCR quantification assays is on the same strand as the mismatch primer and not on the opposite strand. It has been found that in so incorporating the probe in a PCR reaction, additional allele specific discrimination can be provided.

As an example, a TAQMAN™ probe is a hydrolysis probe that has a FAM™ or VIC® dye label on the 5′ end, and minor groove binder (MGB) non-fluorescent quencher (NFQ) on the 3′ end. The TAQMAN™ probe principle generally relies on the 5′-3′ exonuclease activity of Tag® polymerase to cleave the dual-labeled TAQMAN™ probe during hybridization to a complementary probe-binding region and fluorophore-based detection. TAQMAN™ probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-strand DNA-specific dye SYBR® Green (Molecular Probes). SYBR Green is a dye that binds the minor groove of double stranded DNA. When SYBR Green dye binds to a double stranded DNA, the fluorescence intensity increases. As more double stranded amplicons are produced, SYBR Green dye signal will increase.

In any one of the methods provided herein the PCR may be digital PCR. Digital PCR involves partitioning of diluted amplification products into a plurality of discrete test sites such that most of the discrete test sites comprise either zero or one amplification product. The amplification products are then analyzed to provide a representation of the frequency of the selected genomic regions of interest in a sample. Analysis of one amplification product per discrete test site results in a binary “yes-or-no” result for each discrete test site, allowing the selected genomic regions of interest to be quantified and the relative frequency of the selected genomic regions of interest in relation to one another be determined. In certain aspects, in addition to or as an alternative, multiple analyses may be performed using amplification products corresponding to genomic regions from predetermined regions. Results from the analysis of two or more predetermined regions can be used to quantify and determine the relative frequency of the number of amplification products. Using two or more predetermined regions to determine the frequency in a sample reduces a possibility of bias through, e.g., variations in amplification efficiency, which may not be readily apparent through a single detection assay. Methods for quantifying DNA using digital PCR are known in the art and have been previously described, for example in U.S. Patent Publication number US20140242582.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrccoccus furiosus (Pfu), Pyrccoccus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl2, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg²⁺] is too low, a PCR product may not form. If the concentration of magnesium [Mg²⁺] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In some embodiments, the primers used in accordance with the disclosure are modified. The primers may be designed to bind with high specificity to only their intended target (e.g., a particular SNV) and demonstrate high discrimination against further nucleotide sequence differences. The primers may be modified to have a particular calculated melting temperature (Tm), for example a melting temperature ranging from 46° C. to 64° C. To design primers with desired melting temperatures, the length of the primer may be varied and/or the GC content of the primer may be varied. Typically, increasing the GC content and/or the length of the primer will increase the Tm of the primer. Conversely, decreasing the GC content and/or the length of the primer will typically decrease the Tm of the primer. It should be appreciated that the primers may be modified by intentionally incorporating mismatch(es) with respect to the target in order to detect a particular SNV (or other form of sequence non-identity) over another with high sensitivity. Accordingly, the primers may be modified by incorporating one or more mismatches with respect to the specific sequence (e.g., a specific SNV) that they are designed to bind.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a PCR assay can be performed as provided herein or as otherwise would be apparent to one of ordinary skill in the art. As an example, amplification traces are analyzed for consistency and robust quantification. Internal standards may be used to translate the cycle threshold to amount of input nucleic acids (e.g., DNA). The amounts of alleles can be computed as the mean of performant assays and can be adjusted for genotype. A wide range of efficient amplifications shows successful detection of low concentration nucleic acids.

It has been found that the methods and compositions provided herein can be used to detect low-level nucleic acids, such as non-native nucleic acids, in a sample. Accordingly, the methods provided herein can be used on samples where detection of relatively rare nucleic acids is needed. In some embodiments, any one of the methods provided herein can be used on a sample to detect non-native nucleic acids that are at least about 0.25% in the sample relative to total nucleic acids, such as total cf-DNA. In some embodiments, any one of the methods provided herein can be used on a sample to detect non-native nucleic acids that are at least about 0.5% in the sample relative to total nucleic acids, such as total cf-DNA. In some embodiments, any one of the methods provided herein can be used on a sample to detect non-native nucleic acids that are at least about 1% in the sample relative to total nucleic acids, such as total cf-DNA. In some embodiments, any one of the methods provided herein can be used on a sample to detect non-native nucleic acids that are at least about 2% in the sample. In some embodiments, any one of the methods provided herein can be used on a sample to detect non-native nucleic acids that are at least about 5% in the sample.

Because of the ability to determine amounts of non-native nucleic acids, even at low levels, the methods and compositions provided herein can be used to assess a risk in a subject, such as a cancer in the subject. A “risk” as provided herein, refers to the presence or absence or progression of any undesirable condition in a subject, or an increased likelihood of the presence or absence or progression of such a condition, e.g., cancer. The cancer can be any one of the cancers provided herein. As provided herein “increased risk” refers to the presence or progression of any undesirable condition in a subject or an increased likelihood of the presence or progression of such a condition. As provided herein, “decreased risk” refers to the absence of any undesirable condition or progression in a subject or a decreased likelihood of the presence or progression (or increased likelihood of the absence or nonprogression) of such a condition.

As provided herein, early detection or monitoring of conditions, such as cancer, can facilitate treatment and improve clinical outcomes. As mentioned above, any one of the methods provided can be performed on a subject with or at risk of having cancer or a tumor or recurrence of cancer or a tumor or metastasis of a cancer or tumor. Accordingly, in some embodiments, the subject is a subject suspected of having cancer, metastasis and/or recurrence of cancer. In some embodiments, the subject may show no signs or symptoms of having a cancer, metastasis, and/or recurrence. However, in some embodiments, the subject may show symptoms associated with cancer. The type of symptoms will depend upon the type of cancer and are well known in the art.

Cancers include, but are not limited to, leukemias, lymphomas, myelomas, carcinomas, metastatic carcinomas, sarcomas, adenomas, nervous system cancers and geritourinary cancers. Exemplary cancers include, but are not limited to, adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, osteosarcoma, fibrous histiocytoma, brain cancer, brain stem glioma, cerebellar astrocytoma, malignant glioma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, and Wilms tumor. In some embodiments, the cancer is prostate cancer, bladder cancer, pancreatic cancer, lung cancer, kidney cancer, breast cancer, or colon cancer.

The risk in a subject can be determined, for example, by assessing the amount of non-native cf-DNA, such as cancer-specific cell-free-DNA (CS cf-DNA). CS cf-DNA refers to DNA that presumably is shed from the cancer, the sequence of which matches (in whole or in part) the genotype of the cancer. As used herein, CS cf-DNA may refer to certain sequence(s) in the CS cf-DNA population, where the sequence is distinguishable from the subject cf-DNA (e.g., having a different sequence at a particular nucleotide location(s)), or it may refer to the entire CS cf-DNA population.

In some embodiments, any one of the methods provided herein can comprise correlating an increase in non-native nucleic acids and/or an increase in the ratio, or percentage, of non-native nucleic acids relative to native nucleic acids or total nucleic acids, with an increased risk of a condition, such as cancer. In some embodiments of any one of the methods provided herein, correlating comprises comparing a level (e.g., concentration, ratio or percentage) of non-native nucleic acids to a threshold value to identify a subject at increased or decreased risk of a condition. In some embodiments of any one of the methods provided herein, a subject having an increased amount of non-native nucleic acids compared to a threshold value is identified as being at increased risk of a condition. In some embodiments of any one of the methods provided herein, a subject having a decreased or similar amount of non-native nucleic acids compared to a threshold value is identified as being at decreased risk of a condition.

As used herein, “amount” refers to any quantitative value for the measurement of nucleic acids and can be given in an absolute or relative amount. Further, the amount can be a total amount, frequency, ratio, percentage, etc. As used herein, the term “level” can be used instead of “amount” but is intended to refer to the same types of values. In some preferred embodiments of any one of the methods provided herein, the total amount of nucleic acids is determined by a MOMA assay as provided herein and is a measure of native and non-native nucleic acid counts as determined by the MOMA assay, preferably, from informative targets. In some embodiments, the total amount of nucleic acids is determined by any method such as a MOMA assay as provided herein or other assays known to those of ordinary skill in the art but not a MOMA assay as provided herein.

“Threshold” or “threshold value”, as used herein, refers to any predetermined level or range of levels that is indicative of the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a condition or risk or after a course of treatment. Such a baseline can be indicative of a normal or other state in the subject not correlated with the risk or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art.

Changes in the levels of non-native nucleic acids can also be monitored over time. For example, a change from a threshold value (such as a baseline) in the amount, such as ratio or percentage, of non-native nucleic acids can be used as a non-invasive clinical indicator of risk, e.g., risk associated with cancer. This can allow for the measurement of variations in a clinical state and/or permit calculation of normal values or baseline levels. Generally, as provided herein, the amount or level, such as the ratio or percent, of non-native nucleic acids can be indicative of the presence or absence of a risk associated with a condition, such as cancer, or can be indicative of the need for further testing or surveillance. In one embodiment of any one of the methods provided herein, the method may further include an additional test(s) for assessing a condition, such as cancer, etc. The additional test(s) may be any one of the methods provided herein.

In some embodiments of any one of the methods provided herein, where a non-native nucleic acid amount or level, such as ratio or percentage, is determined to be above a threshold value, any one of the methods provided herein can further comprise performing another test on the subject or sample therefrom. Such other tests can be any other test known by one of ordinary skill in the art to be useful in determining the presence or absence of a risk, e.g., in a subject having, at risk of having, or suspected of having cancer, progressing cancer, a metastasis or recurrence of cancer, etc. In some embodiments, the other test is any one of the methods provided herein.

Exemplary additional tests for subjects suspected of having cancer, metastasis, and/or recurrence, include, but are not limited to, biopsy (e.g., fine-needle aspiration, core biopsy, or lymph node removal), X-ray, CT scan, ultrasound, MRI, endoscopy, circulating tumor cell levels, complete blood count, detection of specific tumor biomarkers (e.g., EGFR,ER, HER2, KRAS, c-KIT, CD20, CD30, PDGFR, BRAF, or PSMA), and/or genotyping (e.g., BRCA1, BRCA2, HNPCC, MLH1, MSH2, MSH6, PMS1, or PMS2). The type of additional test(s) will depend upon the type of suspected cancer/metastasis/recurrence and is well within the determination of the skilled artisan.

In some embodiments, the method may further comprise further testing or recommending further testing to the subject and/or treating or suggesting treatment to the subject. In some of these embodiments, the further testing is any one of the methods provided herein. In some of these embodiments, the treating is a cancer treatment. In some embodiments, the information is provided in written form or electronic form. In some embodiments, the information may be provided as computer-readable instructions. In some embodiments, the information may be provided orally.

As provided herein, any one of the methods provided can include a step of providing a therapy or information regarding a therapy to a subject. The therapies can be for treating cancer, a tumor or metastasis, such as an anti-cancer therapy. Such therapies include, but are not limited to, antitumor agents, such as docetaxel; corticosteroids, such as prednisone or hydrocortisone; immunostimulatory agents; immunomodulators; or some combination thereof. Antitumor agents include cytotoxic agents, chemotherapeutic agents and agents that act on tumor neovasculature. Cytotoxic agents include cytotoxic radionuclides, chemical toxins and protein toxins. The cytotoxic radionuclide or radiotherapeutic isotope can be an alpha-emitting or beta-emitting. Cytotoxic radionuclides can also emit Auger and low energy electrons. Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents include dolastatins (U.S. Pat. Nos. 6,034,065 and 6,239,104) and derivatives thereof. Toxins also include poisonous lectins, plant toxins such as ricin, abrin, modeccin, botulina and diphtheria toxins. Other chemotherapeutic agents are known to those skilled in the art. Examples of cancer chemotherapeutic agents include, but are not limited to, irinotecan (CPT-11); erlotinib; gefitinib (Iressa™); imatinib mesylate (Gleevec); oxalipatin; anthracyclins-idarubicin and daunorubicin; doxorubicin; alkylating agents such as melphalan and chlorambucil; cis-platinum, methotrexate, and alkaloids such as vindesine and vinblastine. In some embodiments, further or alternative cancer treatments are contemplated herein, such as radiation and/or surgery.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are well known in the art (see, e.g., Remington's Pharmaceutical Sciences by E. W. Martin).

Any one of the methods provided herein can comprise extracting nucleic acids, such as cell-free DNA, from a sample obtained from a subject. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

As used herein, the sample from a subject can be a biological sample. Examples of such biological samples include whole blood, plasma, serum, urine, etc. In some embodiments, addition of further nucleic acids, e.g., a standard, to the sample can be performed.

In some embodiments of any one of the methods provided herein, a pre-amplification step is performed. An exemplary method of such an amplification is as follows, and such a method can be included in any one of the methods provided herein. Approximately 15 ng of cell-free plasma DNA is amplified in a PCR using Q5 DNA polymerase with approximately 13 targets where pooled primers were at 4 uM total. Samples undergo approximately 25 cycles. Reactions are in 25 ul total. After amplification, samples can be cleaned up using several approaches including AMPURE bead cleanup, bead purification, or simply ExoSAP-IT™, or Zymo.

The present disclosure also provides compositions or kits that can be useful for assessing an amount of non-native nucleic acids in a sample. In some embodiments, the composition or kit comprises one or more primer pairs. Each of the primer pairs of the composition or kit can comprise a forward and a reverse primer, wherein there is a 3′ mismatch in one of the primers (e.g., at the penultimate 3′ nucleotide) in some embodiments of any one of the methods, compositions or kits provided herein. In some embodiments of any one of the methods, compositions or kits provided herein, this mismatch is at a 3′ nucleotide and adjacent to the SNV position and when the particular SNV is not present there is a double mismatch with respect to the other allele of the SNV target. In some embodiments of any one of the methods, compositions or kits provided herein, the mismatch primer of a primer pair is the forward primer. In some embodiments of any one of the methods, compositions or kits provided herein, the reverse primer for each allele of a SNV target is the same.

In some embodiments of any one of the compositions or kits provided herein, the composition comprises at least 1, 2, 3, 4, 5, 6, 7, 8, 9, 10, 11, 12, 13, 14, 15, 20, 25, 30 etc. such primer pairs. In some embodiments of any one of the compositions or kits provided herein, the composition comprises at least two primer pairs for each of at least 1, 2, 3, 4, 5, 6, 7, 8, 9, 10, 11, 12, 13, 14, 15 or more targets, such as SNV targets (e.g., biallelic SNV targets). In some embodiments of any one of the compositions or kits provided herein, the composition or kit comprises at least one, such as two primer pairs, for more than 1, 2, 3, 4, 5, 6, 7, 8, 9 or 10 but less than 15 SNV targets. In some embodiments of any one of the compositions or kits provided herein, the primer pairs of the composition or kit are designed to be compatible for use in amplification-based quantification assay, such as a quantitative PCR assay. For example, the primer pairs are designed to prevent primer dimers and/or limit the number of off-target binding sites. It should be appreciated that the primer pairs of the composition or kit may be optimized or designed in accordance with any one of the methods described herein.

In some embodiments, any one of the compositions or kits provided further comprises a buffer. In some embodiments, the buffers contain additives such as surfactants, dimethyl sulfoxide (DMSO), glycerol, bovine serum albumin (BSA) and polyethylene glycol (PEG) or other PCR reaction additive. In some embodiments, any one of the compositions or kits provided further comprises a polymerase for example, the composition or kit may comprise E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. In some embodiments, any one of the compositions or kits provided further comprises one or more dNTPs (e.g., dATP, dCTP, dGTP, dTTP). In some embodiments, any one of the compositions or kits provided further comprises a probe (e.g., a TAQMAN™ probe).

A “kit,” as used herein, typically defines a package or an assembly including one or more of the compositions of the invention, and/or other compositions associated with the invention, for example, as previously described. Any one of the kits provided herein may further comprise at least one reaction tube, well, chamber, or the like. Any one of the primers, primer systems (such as a set of primers for a plurality of targets) or primer compositions described herein may be provided in the form of a kit or comprised within a kit.

Each of the compositions of the kit may be provided in liquid form (e.g., in solution), in solid form (e.g., a dried powder), etc. A kit may, in some cases, include instructions in any form that are provided in connection with the compositions of the invention in such a manner that one of ordinary skill in the art would recognize that the instructions are to be associated with the compositions of the invention. The instructions may include instructions for performing any one of the methods provided herein. The instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—MOMA cf-DNA Assay

This exemplary assay is designed to determine the percentage of CS cf-DNA present in a subject's blood sample. In this embodiment, the subject's blood sample is collected in an EDTA tube and centrifuged to separate the plasma and buffy coat. The plasma and buffy coat can be aliquoted into two separate 15 mL conical tubes and frozen. The plasma sample can be used for quantitative genotyping (qGT), while the buffy coat can be used for basic genotyping (bGT) of the subject.

The first step in the process can be to extract cell free DNA from the plasma sample (used for qGT) and genomic DNA (gDNA) from the buffy coat, whole blood, or tissue sample (used for bGT). The total amount of cfDNA can be determined by qPCR and normalized to a target concentration. This process is known as a cf-DNA Quantification. gDNA can be quantified using UV-spectrophotometry and normalized. Fifteen ng of DNA generally provides accurate and valid results.

The normalized patient DNA can be used as an input into a multiplexed library PCR amplification reaction containing primer pairs, each of which amplify a region including one of the MOMA target sites. The resulting library can be used as the input for either the bGT or qGT assay as it consists of PCR amplicons having the MOMA target primer and probe sites. This step can improve the sensitivity of the overall assay by increasing the copy number of each target prior to the highly-specific qPCR amplification. Controls and calibrators/standards can be amplified with the multiplex library alongside patient samples. Following the library amplification, an enzymatic cleanup can be performed to remove excess primers and unincorporated deoxynucleotide triphosphates (dNTPs) to prevent interference with the downstream amplification.

In a parallel workflow the master mixes can be prepared and transferred to a 384-well PCR plate. The amplified samples, controls, and calibrators/standards can then be diluted with the library dilution buffer to a predetermined volume and concentration. The diluted samples and controls can be aliquoted to a 6-well reservoir plate and transferred to the 384-well PCR plate using an acoustic liquid handler. The plate can then be sealed and moved to a real-time PCR amplification and detection system.

MOMA can perform both the basic and quantitative genotyping analyses by targeting biallelic SNVs that are likely to be distinct between a subject with cancer, and those without, making them highly informative. MOMA assays can be designed to target the tumor-specific SNVs of a patient, or to commonly found tumor-specific SNVs. The quantitative genotyping analysis, along with standard curves, can quantitate to the tumor-specific allele presence for each target, known as a minor-species proportion. The tumor may be heterogeneous and carry multiple SNVs or a single tumor-specific allele. The quality-control passed allele ratios can be used to determine the % of CS cfDNA for each SNV. The results from each informative target can be averaged, in some embodiments.

### Example 2—MOMA Assay with Metastatic Pancreatic Cancer Samples Generation of Multiplex Libraries

Multiplex libraries for 13 cancer-specific targets, including targets for KRAS and p53, were prepared in a 25 cycle reaction using approximately 15 ng of input template DNA and Q5 high-fidelity DNA polymerase (New England Biolabs), resulting in a final concentration of 4 μM pancreatic cancer primer. The cycling protocol was as follows: 1 cycle at 98° C. for 30 seconds; 25 cycles of 98° C. for 10 seconds, 60° C. for 20 seconds, 72° C. for 30 seconds; one cycle at 72° C. for 2 minutes; and then the reaction was stored at 4° C. Reactions were cleaned up with ExoSAP-IT™ (Thermo Fisher Scientific) and overlayed 1:1 with a 1× preservation solution (0.18× TBE and 0.2 μg/ul BSA).

### Human KRAS Exon 2 Quantitative Genotyping Assay

Cell-free DNA (cf-DNA) was isolated from two metastatic pancreatic cancer patients and a healthy control. A spike-in control template; TAI5 was added as a 99:1 (VV:RR) mix at 4500 copies per reaction. Cell line genomic DNA (gDNA) (HD272; Horizon Diagnostics, Cambridge, UK) was used for the mutant human KRAS exon 2 variant DNA (50% mutant DNA). Together with wild type (WT) DNA (100% WT DNA), the two were used to make mixed human genomic DNA samples. These reconstructed DNAs were sonicated to simulate cell free DNA (cf-DNA) and generate theoretical allelic frequencies ranging between 0.25% and 50.0%.

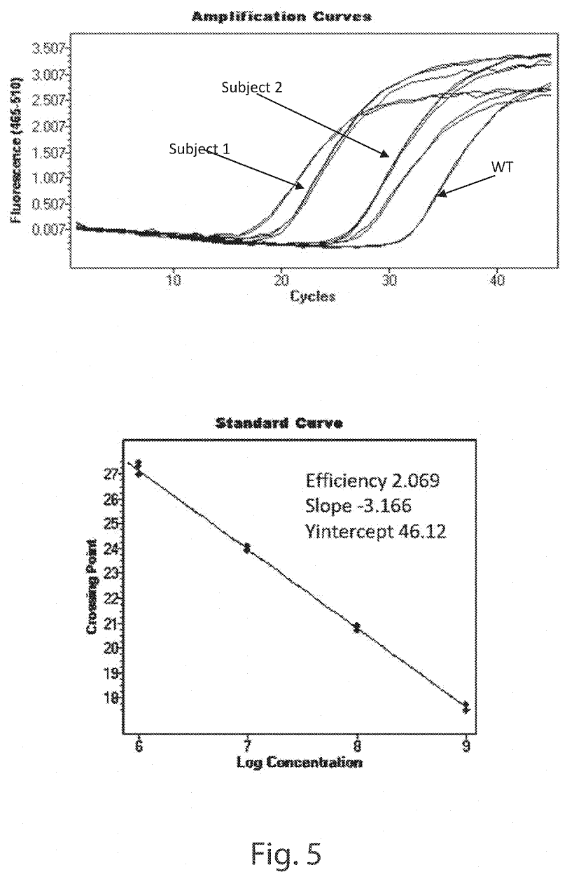

Quantitative genotyping was set up in a 3 μl reaction (1 μl sample; 2 μl master mix) using an AptaTAQ master mix (Roche). Two primer pairs were used for a KRAS exon 2 target (one pair for the variant version specific for cancer and the other pair for the reference KRAS exon 2 sequence). Samples were diluted 1:100 with 0.5× preservation solution (described above). Plates were heat sealed, spun 3 minutes at room temperature and underwent PCR (LC480, Roche) using standard protocols known in the art. The results of the experiment are shown in Table 1 and FIG. 5.

TABLE 1
KRAS Exon 2 Target MOMA Results

| Recon/Patient | Target MOMA |
|---|---|
| (theoretical percentage) | (cancer-specific %) |
| 0.000% | 0.183% |
| 0.250% | 0.289% |
| 0.500% | 0.689% |
| 1.000% | 1.361% |
| 5.000% | 4.778% |
| 10.000% | 9.825% |
| 50.000% | 50.277% |
| WT cfDNA | 0.131% |
| Patient 1 cfDNA | 20.377% |
| Patient 2 cfDNA | 2.058% |

### Example 3—Examples of Computer-Implemented Embodiments

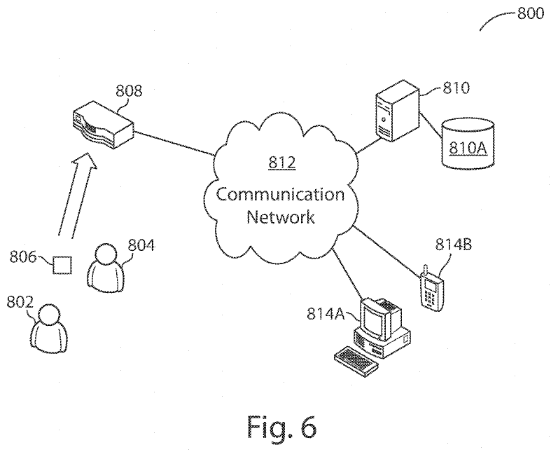

In some embodiments, the diagnostic techniques described above may be implemented via one or more computing devices executing one or more software facilities to analyze samples for a subject over time, measure nucleic acids (such as cell-free DNA) in the samples, and produce a diagnostic result based on one or more of the samples. FIG. 6 illustrates an example of a computer system with which some embodiments may operate, though it should be appreciated that embodiments are not limited to operating with a system of the type illustrated in FIG. 6.

The computer system of FIG. 6 includes a subject 802 and a clinician 804 that may obtain a sample 806 from the subject 802. As should be appreciated from the foregoing, the sample 806 may be any suitable sample of biological material for the subject 802 that may be used to measure the presence of nucleic acids (such as cell-free DNA) in the subject 802, including a blood sample. The sample 806 may be provided to an analysis device 808, which one of ordinary skill will appreciate from the foregoing will analyze the sample 806 so as to determine (including estimate) a total amount of nucleic acids (such as cell-free DNA) and an amount of a non-native nucleic acids (such as cell-free DNA) in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of nucleic acids (such as cell-free DNA) in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of nucleic acids (such as cell-free DNA) in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

The diagnostic facility may receive the result of the analysis and the information describing or identifying the sample 806 and may store that information in the data store 810A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition. The diagnostic facility may determine the diagnosis using any of the various examples described above, including by comparing the amounts of nucleic acids (such as cell-free DNA) determined for a particular sample 806 to one or more thresholds or by comparing a change over time in the amounts of nucleic acids (such as cell-free DNA) determined for samples 806 over time to one or more thresholds. For example, the diagnostic facility may determine a risk to the subject 802 of a condition by comparing an amount of nucleic acids (such as non-native cell-free DNA) for one or more samples 806 to a threshold. Based on the comparisons to the thresholds, the diagnostic facility may produce an output indicative of a risk to the subject 802 of a condition.

As should be appreciated from the foregoing, in some embodiments, the diagnostic facility may be configured with different thresholds to which amounts of nucleic acids (such as cell-free DNA) may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds against which amounts of nucleic acids (such as cell-free DNA) are to be compared, with different thresholds stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition for which a risk is to be determined, and the diagnostic facility may prior to determining the risk receive as input a condition and use the condition to select the thresholds on which to base the determination of risk. It should be appreciated that the diagnostic facility is not limited to selecting thresholds in any particular manner, in embodiments in which multiple thresholds are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of nucleic acids (such as cell-free DNA) detected in one or more samples 806 for a subject 802. In some embodiments, user interfaces may include any of the examples of results, values, amounts, graphs, etc. discussed above. They can include results, values, amounts, etc. over time. For example, in some embodiments, a user interface may incorporate a graph similar to that shown in any one of the figures provided herein. In such a case, in some cases the graph may be annotated to indicate to a user how different regions of the graph may correspond to different diagnoses that may be produced from an analysis of data displayed in the graph. For example, thresholds against which the graphed data may be compared to determine the analysis may be imposed on the graph(s).

A user interface including a graph, particularly with the lines and/or shading, may provide a user with a far more intuitive and faster-to-review interface to determine a risk of the subject 802 based on amounts of nucleic acids (such as cell-free DNA), than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of nucleic acids (such as cell-free DNA). The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

What is claimed is:

1. A method of assessing an amount of cancer-specific nucleic acids in a sample from a subject, the method comprising:
for each of one or more single nucleotide variant (SNV) targets, performing an amplification-based quantification assay, such as a polymerase chain reaction (PCR) quantification assay, on the sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target in a primer and specifically amplifies the one allele of the SNV target, and another of the at least two primer pairs specifically amplifies the another allele of the SNV target,
and obtaining or providing results from the amplification-based quantification assays, such as PCR quantification assays, to determine the amount of cancer-specific nucleic acids in the sample.

2. The method of claim 1, wherein the results are provided in a report.

3. The method of claim 1 or 2, wherein the method further comprises determining the amount of the cancer-specific nucleic acids in the sample based on the results.

4. The method of claim 1 or 2, wherein the results comprise the amount of the cancer-specific nucleic acids in the sample.

5. A method of assessing an amount of cancer-specific nucleic acids in a sample from a subject, the method comprising:
obtaining results from an amplification-based quantification assay, such as a polymerase chain reaction (PCR) quantification assay, for each of one or more single nucleotide variant (SNV) targets, performed on the sample, or portion thereof, with at least two primer pairs, wherein each primer pair comprises a forward primer and a reverse primer, wherein one of the at least two primer pairs comprises a 3′ penultimate mismatch relative to one allele of the SNV target but a 3′ double mismatch relative to another allele of the SNV target and specifically amplifies the one allele of the SNV target in a primer, and another of the at least two primer pairs specifically amplifies the another allele of the SNV target, and
assessing the amount of cancer-specific nucleic acids based on the results.

6. The method of claim 5, wherein the amount of the cancer-specific nucleic acids in the sample is based on the results of the amplification-based quantification assays, such as PCR quantification assays.

7. The method of claim 5 or 6, wherein the results are obtained from a report.

8. The method of any one of the preceding claims, wherein the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

9. The method of any one of the preceding claims, wherein the amount is the ratio or percentage of cancer-specific nucleic acids to wild-type or total nucleic acids as measured in the assay.

10. The method of any one of the preceding claims, wherein the results are informative results of the amplification-based quantification assays, such as PCR quantification assays.

11. The method of any one of the preceding claims, wherein the amount is based on informative results of the amplification-based quantification assays, such as PCR quantification assays.

12. The method of any one of the preceding claims, wherein the method further comprises selecting informative results of the amplification-based quantification assays, such as PCR quantification assays.

13. The method of claim 12, wherein the selected informative results are averaged.

14. The method of claim 12 or 13, wherein the informative results of the amplification-based quantification assays, such as PCR quantification assays, are selected based on the genotype of the subject.

15. The method of any one of the preceding claims, wherein the method further comprises obtaining the genotype of the subject.

16. The method of any one of the preceding claims, wherein the method further comprises obtaining the plurality of SNV targets.

17. The method of any one of the preceding claims, wherein the method further comprises obtaining the at least two primer pairs for each of the one or more SNV targets.

18. The method of any one of the preceding claims, wherein the one or more SNV targets is at least 1 SNV target.

19. The method of any one of the preceding claims, wherein the one or more SNV targets is at least 2 SNV targets.

20. The method of any one of the preceding claims, wherein the one or more SNV targets is at least 3 SNV targets.

21. The method of any one of the preceding claims, wherein the one or more SNV targets is at least 4 SNV targets.

22. The method of any one of the preceding claims, wherein the one or more SNV targets is at least 5, 6, 7, 8, 9, 10, 11, 12, 13, 14 or 15 SNV targets.

23. The method of any one of the preceding claims, wherein the one or more SNV targets are each specific to the same kind of cancer.

24. The method of any one of claims 1-22, wherein one or more of the one or more SNV targets is specific to pancreatic cancer.

25. The method of any one of the preceding claims, wherein the one or more SNV targets comprise a SNV target in the KRAS gene and/or p53 gene.

26. The method of any one of claims 1-22, wherein the one or more SNV targets are each specific to a cancer in the subject.

27. The method of claim 26, wherein the method further comprises obtaining the genotype of the cancer in the subject.

28. The method of any one of claims 19-22, wherein at least one SNV target is specific to one kind of cancer and at least one other SNV target is specific to another kind of cancer.

29. The method of any one of claims 19-22, wherein the one or more SNV targets are specific to more than 1, 2, 3, 4 or 5 kinds of cancer or derived from more than 1, 2, 3, 4 or 5 genes associated with cancer-specific mutations.

30. The method of any one of the preceding claims, wherein the amount of cancer-specific nucleic acids in the sample is at least 0.25%.

31. The method of claim 30, wherein the amount of cancer-specific nucleic acids in the sample is at least 0.5%.

32. The method of claim 31, wherein the amount of cancer-specific nucleic acids in the sample is at least 0.75%.

33. The method of claim 32, wherein the amount of cancer-specific nucleic acids in the sample is at least 1%.

34. The method of claim 33, wherein the amount of cancer-specific nucleic acids in the sample is at least 2%.

35. The method of claim 34, wherein the amount of cancer-specific nucleic acids in the sample is at least 5%.

36. The method of any one of the preceding claims, wherein the cancer-specific nucleic acids are cancer-specific cell-free DNA.

37. The method of any one of the preceding claims, wherein the PCR quantification assays are real time PCR assays or digital PCR assays.

38. The method of any one of the preceding claims, wherein the method further comprises determining a risk in the subject based on the amount of cancer-specific nucleic acids in the sample.

39. The method of claim 38, wherein the risk is a risk associated with cancer.

40. The method of claim 38 or 39, wherein the risk is increased if the amount of cancer-specific nucleic acids is greater than a threshold value.

41. The method of claim 38 or 39, wherein the risk is decreased if the amount of cancer-specific nucleic acids is less than a threshold value.

42. The method of any one of the preceding claims, wherein the method further comprises selecting a treatment for the subject based on the amount of cancer-specific nucleic acids.

43. The method of any one of the preceding claims, wherein the method further comprises treating the subject based on the amount of cancer-specific nucleic acids.

44. The method of any one of the preceding claims, wherein the method further comprises providing information about a treatment to the subject based on the amount of cancer-specific nucleic acids.

45. The method of any one of the preceding claims, wherein the method further comprises monitoring or suggesting the monitoring of the amount of cancer-specific nucleic acids in the subject over time or at a subsequent point in time.

46. The method of any one of the preceding claims, wherein the method further comprises evaluating an effect of a treatment administered to the subject based on the amount of cancer-specific nucleic acids.

47. The method of any one of claims 42-46, wherein the treatment is a cancer treatment.

48. The method of any one of the preceding claims, further comprising providing or obtaining the sample or a portion thereof.

49. The method of any one of the preceding claims, further comprising extracting nucleic acids from the sample.

50. The method of any one of the preceding claims, further comprising performing a pre-amplification step.

51. The method of any one of the preceding claims, wherein the sample comprises blood, plasma or serum.

52. A composition or kit comprising,
primer pair, for each of one or more cancer-specific SNV targets, wherein each primer pair comprises a 3′ penultimate mismatch relative to one allele of a SNV target but a 3′ double mismatch relative to another allele of the SNV target in a primer and specifically amplifies the one allele of the SNV target, wherein the one or more SNV targets.

53. The composition or kit of claim 52, further comprising another primer pair for each of the one or more cancer-specific SNV targets wherein the another primer pair specifically amplifies the another allele of the SNV target.

54. The composition or kit of claim 52 or 53, wherein the one or more cancer-specific SNV targets is at least 2, 3, 4, 5, 6, 7, 8, 9, 10, 11, 12, 13, 14 or 15 SNV targets.

55. The composition or kit of any one of claims 52-54, wherein the cancer-specific SNV targets are each specific to the same kind of cancer.

56. The composition or kit of any one of claims 52-54, wherein one or more of the one or more SNV targets is specific to pancreatic cancer.

57. The composition or kit of any one of claims 52-54, wherein the cancer-specific SNV targets comprise a SNV target in the KRAS gene and/or p53 gene.

58. The composition or kit of any one of claims 52-54, wherein the cancer-specific SNV targets are each specific to a cancer in the subject.

59. The composition or kit of any one of claims 52-54, wherein at least one SNV target is specific to one kind of cancer and at least one other SNV target is specific to another kind of cancer.

60. The composition or kit of any one of claims 52-54, wherein the one or more SNV targets are specific to more than 1, 2, 3, 4 or 5 kinds of cancer or derived from more than 1, 2, 3, 4 or 5 genes associated with cancer-specific mutations.

61. The composition or kit of any one of claims 53-60, wherein the another primer pair for each of the SNV targets also comprises a 3′ penultimate mismatch relative to the another allele of the SNV target but a 3′ double mismatch relative to the one allele of the SNV target in a primer and specifically amplifies the another allele of the SNV target.

62. The composition or kit of any one of claims 52-61, further comprising a buffer.

63. The composition or kit of any one of claims 52-62, further comprising a polymerase.

64. The composition or kit of any one of claims 52-63, further comprising a probe.

65. The composition or kit of claim 64, wherein the probe is a fluorescent probe.

66. The composition or kit of any one of claims 52-65, further comprising instructions for use.

67. The composition or kit of claim 66, wherein the instructions for use are instructions for determining or assessing the amount of cancer-specific nucleic acids in a sample from a subject with cancer or suspected of having cancer.

68. The composition or kit of any one of claims 52-67 for use in a method of any one of claims 1-51.

69. The composition or kit of any one of claims 52-67, for use in any one of the methods provided herein.

70. A method comprising:
obtaining the amount of cancer-specific nucleic acids based on the method of any one of claims 1-51, and
assessing a risk in a subject that is at risk of cancer, has cancer, is suspected of having cancer or previously had cancer based on the levels or amount.

71. The method of claim 70, wherein a treatment or information about a treatment or non-treatment is selected for or provided to the subject based on the assessed risk.

72. The method of claim 70 or 71, wherein the method further comprises monitoring or suggesting the monitoring of the amount of cancer-specific nucleic acids in the subject over time.
